(19) United States
(12) Patent Application Publication
(10) Pub. No.: US 2026/0165671 A1
(43) Pub. Date: Jun. 18, 2026

(54) X-RAY COMPUTED TOMOGRAPHY APPARATUS AND INFORMATION PROCESSING METHOD

(71) Applicant: CANON MEDICAL SYSTEMS CORPORATION, Otawara-shi (JP)

(72) Inventor: Ryusei SAIKI, Nasushiobara (JP)

(73) Assignee: CANON MEDICAL SYSTEMS CORPORATION, Otawara-shi (JP)

(21) Appl. No.: 19/412,973

(22) Filed: Dec. 9, 2025

(30) Foreign Application Priority Data
Dec. 13, 2024 (JP) 2024-218779

(51) Int. Cl. A61B 6/00; A61B 6/03; A61B 6/04

(52) U.S. Cl. CPC A61B 6/547; A61B 6/035; A61B 6/0407; A61B 6/542

(57) ABSTRACT

An X-ray computed tomography (CT) apparatus according to an embodiment includes processing circuitry configured to identify gantry state information indicating a state of a gantry including an opening, the gantry state information including at least one of posture information indicating a posture of the gantry and object information indicating an object other than a subject present in the opening, determine a dose parameter used to calculate an output dose based on the identified gantry state information and parameter information to be stored by associating the gantry state information with the dose parameter used to calculate the output dose to be output from the X-ray CT apparatus, and calculate the output dose using the determined dose parameter.

### CROSS-REFERENCE TO RELATED APPLICATIONS

This application is based upon and claims the benefit of priority from Japanese Patent Application No. 2024-218779, filed Dec. 13, 2024, the entire contents of which are incorporated herein by reference.

### FIELD

Embodiments described herein relate to an X-ray computed tomography (CT) apparatus and an information processing method.

### BACKGROUND

An X-ray computed tomography (CT) apparatus configured to capture an image of a subject in a lying-position state or a standing-position state has heretofore been known.

An X-ray CT apparatus is configured to calculate an exposure dose output from the X-ray CT apparatus (the exposure dose is also referred to as an “output dose”) and output the output dose to a user interface, an examination summary, exposure dose structured reporting (DoseSR), or the like. Data to be output to the user interface, examination summary, DoseSR, or the like is adjusted to an optimum value for each apparatus by calculations using dose parameters.

In an X-ray CT apparatus configured to capture images of a subject in a lying position state and a standing position state, a couch (couchtop) is generally used to support the subject in the lying position state, and a columnar subject holder (also referred to as a subject fixing pole) is generally used to support the subject in the standing position state.

Thus, an object other than the subject that is present in an opening of a gantry during imaging in a lying-position mode is different from an object other than the subject that is present in the opening of the gantry during imaging in a standing-position mode (or a seated-position mode), to be more specific, the couchtop is present in the opening of the gantry during imaging in the lying-position mode, while the subject fixing pole is present in the opening of the gantry during imaging in the standing-position mode (or the seated-position mode). For this reason, the amount of absorption of X-rays in the subject during imaging in the lying-position mode is different from that during imaging in the standing-position mode. In other words, an exposure dose of the subject during imaging in the lying-position mode is different from that during imaging in the standing-position mode.

In general, one dose parameter is set for one X-ray CT apparatus. Accordingly, even in a case where the same imaging conditions are used by the same X-ray CT apparatus, there is a possibility that data to be output to a user interface, an examination summary, DoseSR, or the like cannot be adjusted to an optimum value depending on an imaging mode.

### BRIEF DESCRIPTION OF THE DRAWINGS

FIG. 1 illustrates a configuration example of an X-ray computed tomography (CT) apparatus according to an embodiment;

FIG. 2 is a perspective view illustrating an example of a state of a gantry apparatus in a standing-position mode according to the embodiment;

FIG. 3 is a perspective view illustrating an example of the state of the gantry apparatus in a lying-position mode according to the embodiment;

FIG. 4 illustrates an example of an output dose display screen when an image of a subject is captured in the lying-position mode according to the embodiment;

FIG. 5 illustrates an example of the output dose display screen when an image of the subject is captured in the standing-position mode according to the embodiment; and

FIG. 6 is a flowchart illustrating an example of processing to be executed by the X-ray CT apparatus according to the embodiment.

### DETAILED DESCRIPTION

An X-ray computed tomography (CT) apparatus according to an embodiment of the present disclosure includes an identification unit, a determination unit, and a calculation unit. The identification unit identifies gantry state information that indicates a state of a gantry and includes at least one of posture information indicating a posture of a gantry including an opening and object information indicating an object other than a subject present in the opening. The determination unit determines a dose parameter used to calculate an output dose based on the identified gantry state information and parameter information to be stored by associating the gantry state information with the dose parameter used to calculate the output dose to be output from the X-ray CT apparatus. The calculation unit calculates the output dose using the determined dose parameter.

Various Embodiments will be described hereinafter with reference to the accompanying drawings.

An X-ray CT imaging apparatus and an information processing method according to embodiments will be described below with reference to the drawings. The X-ray CT apparatus according to the present embodiment has a structure in which the posture of the gantry can be changed between a standing-position imaging state in which an image of a subject P can be captured in a standing position and a lying-position imaging state in which an image of the subject P can be captured in a lying position. In the following embodiments, assuming that components denoted by the same reference numerals perform similar operations, redundant descriptions will be omitted as appropriate.

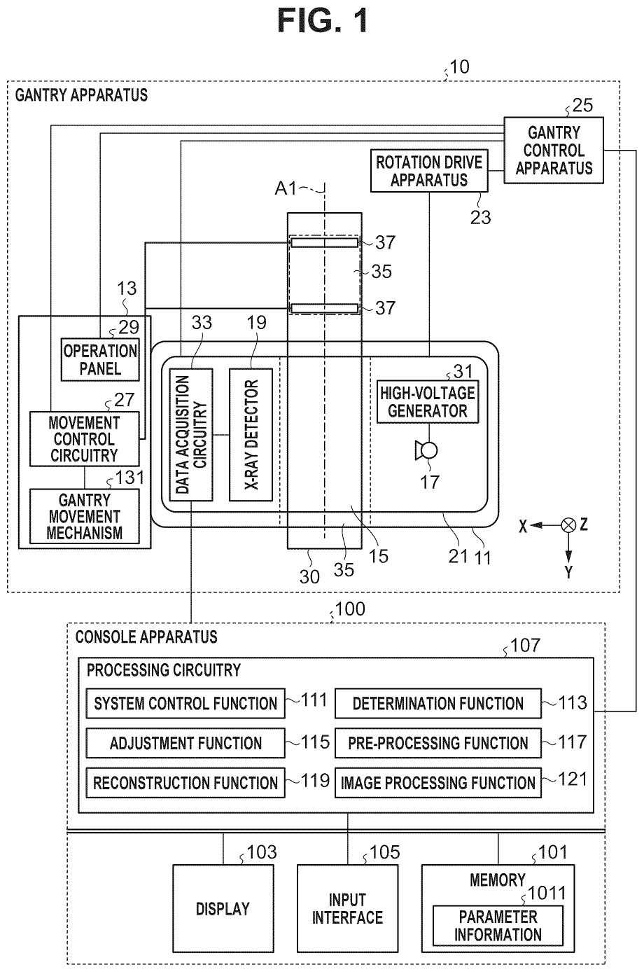

FIG. 1 illustrates a configuration example of an X-ray CT apparatus 1 according to an embodiment. As illustrated in FIG. 1, the X-ray CT apparatus 1 includes a gantry apparatus 10 and a console apparatus 100. For example, the gantry apparatus 10 is installed in a CT examination room, and the console apparatus 100 is installed in a control room that is adjacent to the CT examination room. The gantry apparatus 10 and the console apparatus 100 are connected by a wire or wirelessly so that the gantry apparatus 10 and the console apparatus 100 can communicate with each other.

In the present embodiment, an axial direction perpendicular to a floor surface, that is, a vertical direction, is defined as a Z-axis direction, and two directions that are perpendicular to the Z-axis direction and are perpendicular to each other are defined as an X-axis direction and a Y-axis direction, respectively.

The gantry apparatus 10 is a scanning apparatus configured to perform X-ray CT imaging on the subject P in a standing position state or a lying position state. The console apparatus 100 is a computer that controls the gantry apparatus 10.

The gantry apparatus 10 includes a gantry 11, a post 13, a rotation drive apparatus 23, and a gantry control apparatus 25.

The gantry 11 includes an imaging system related to imaging of the subject P, and an opening 15 into which the subject P can be inserted. The post 13 supports the gantry 11 so that the orientation of the opening 15 can be changed between the vertical direction and the horizontal direction and the gantry 11 can be moved along the vertical direction.

While FIG. 1 illustrates an example where the gantry 11 is supported in a cantilever manner by the post 13, the present embodiment is not limited to this example. For example, the gantry 11 may be supported by a plurality of posts (e.g., two posts). The post 13 may also be referred to as a post portion.

The gantry 11 includes the opening 15 that forms an imaging space related to imaging of the subject P. The gantry 11 is a substantially cylindrical structure provided with the opening 15. As illustrated in FIG. 1, the gantry 11 accommodates an X-ray tube 17 and an X-ray detector 19 that are opposed to each other with respect to the opening 15. The X-ray tube 17 and the X-ray detector 19 are included in the imaging system related to imaging of the subject P according to the present embodiment.

The imaging system may further include data acquisition circuitry (hereinafter also referred to as a data acquisition system (DAS)) 33, a high-voltage generator 31, a collimator, and a wedge. In other words, the gantry 11 includes the imaging system related to imaging of the subject P. The gantry 11 is supported by the post 13 so that the gantry 11 can be moved in the vertical direction along the post 13.

The gantry 11 is supported by the post 13 so that the orientation of the opening 15 can be changed between the vertical direction and the horizontal direction. The orientation of the opening 15 corresponds to, for example, a direction in which the couchtop 30 is inserted in the opening 15, or a direction along a rotation axis A1.

The gantry 11 also includes a main frame (not illustrated) formed of metal such as aluminum, and a rotation frame 21 that is rotatably supported by the main frame about the rotation axis A1 via a bearing or the like. A contact portion between the main frame and the rotation frame 21 is provided with an annular electrode (not illustrated). A conductive slider (not illustrated) is attached to the contact portion of the main frame so that the slider can be brought into sliding contact with the annular electrode.

The post 13 is a substrate that supports the gantry 11 separately from a floor surface.

The post 13 has a columnar shape such as a cylindrical shape or a prismatic shape. The post 13 is formed of any material such as plastic or metal. The post 13 is attached to, for example, a side surface of the gantry 11. The post 13 supports the gantry 11 slidably in the vertical direction in a state where the rotation axis A1 of the opening 15 is substantially perpendicular to the floor surface so as to perform X-ray CT imaging of the subject P in a seated-position posture or a standing-position posture.

Typically, the post 13 is provided on one side of the gantry 11. However, the present embodiment is not limited to this example. For example, two posts 13 may be respectively connected to the both sides of the gantry 11. In other words, at least one post 13 supports the gantry 11 so as to be movable in the vertical direction.

An example where the post 13 has a columnar shape has been described above. However, the present embodiment is not limited to this example. For example, the post 13 may have any shape, such as a U-shape, as long as at least one side of the gantry 11 can be supported.

The post 13 supports the gantry 11 in such a manner that the rotation axis A1 can rotate about a horizontal axis (hereinafter also referred to as a tilt axis) that is parallel to the floor surface between the vertical direction and the horizontal direction. The post 13 and the gantry 11 are connected via, for example, a slewing ring bearing or the like so that the gantry 11 can be rotated about the tilt axis.

Specifically, the post 13 is provided with a linear guide along the vertical direction. A block configured to move along the linear guide is provided with a slewing ring bearing. The block is driven by a motor under the control of movement control circuitry 27, so that the block can move along the linear guide.

A gear fitted to a gear (internal teeth) in the slewing ring bearing is connected to a rotation shaft of the motor via various gears and the like that generate a predetermined torque. The internal teeth in the slewing ring bearing are rotated by being driven by the motor under the control of the movement control circuitry 27.

With the configurations described above, the gantry 11 can be rotated about the X-axis illustrated in FIG. 1 as a rotation axis and can be moved along the vertical direction. The above-described linear guide and slewing ring bearing correspond to a gantry movement mechanism 131 related to the movement of the gantry 11. In other words, the gantry movement mechanism 131 is mounted on the post 13.

The gantry movement mechanism 131 moves the block along the linear guide located along the vertical direction under the control of the movement control circuitry 27, thereby moving the gantry 11. This enables the gantry 11 to move up and down along the vertical direction. A mechanism related to the movement of the gantry 11 along the vertical direction is not limited to a linear guide or the like, but instead may be implemented by an existing mechanism such as a rack and pinion.

The gantry movement mechanism 131 causes the gantry 11 to rotate between the horizontal direction and the vertical direction by the rotation of the internal teeth in the slewing ring bearing under the control of the movement control circuitry 27.

A rotation mechanism that causes the gantry 11 to rotate is not limited to a slewing ring bearing, but instead may be implemented by an existing mechanism. The rotation of the gantry 11 by the rotation mechanism enables switching between a standing-position or seated-position imaging state (also referred to as a standing-position mode) and a lying-position imaging state (also referred to as a lying-position mode), or switching between the standing-position mode and the lying-position mode.

For example, in the case of performing lying-position imaging on the subject P, the gantry movement mechanism 131 causes the gantry 11 to rotate so that the opening 15 is oriented in the vertical direction under the control of the movement control circuitry 27. After the subject P lies down on a couchtop 30, couchtop movement mechanisms 37 to be described below move the couchtop 30 horizontally, thereby enabling lying-position imaging of the subject P, like in the general X-ray CT apparatus.

In the case of performing standing-position imaging on the subject P, the rotation mechanism in the gantry movement mechanism 131 causes the gantry 11 to rotate so that the opening 15 is oriented in the horizontal direction under the control of the movement control circuitry 27. While the subject P stands upright with his or her back leaning against the post 13, standing-position imaging is executed by moving the gantry 11 up and down. In the standing-position mode, the couchtop movement mechanisms 37 to be described below cause the couchtop 30 to evacuate to a position where the couchtop 30 does not interfere with the gantry 11 under the control of the movement control circuitry 27.

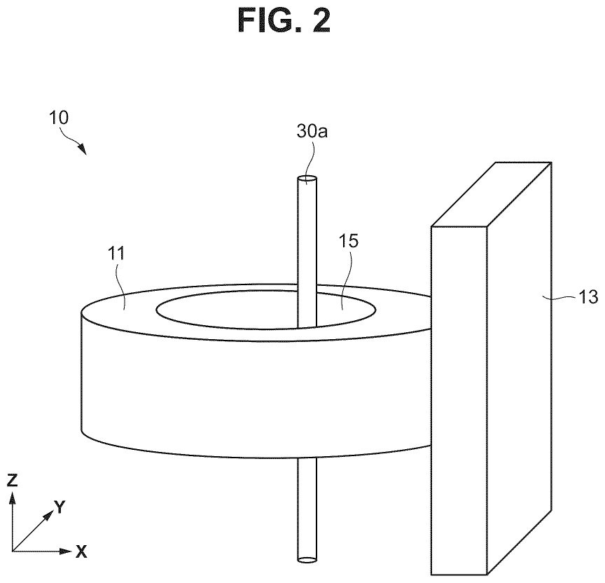

FIG. 2 is a perspective view illustrating an example of the state of the gantry apparatus 10 in the standing-position mode. As illustrated in FIG. 2, according to the present embodiment, in the standing-position mode, standing-position imaging of the subject P is performed in a state where a subject fixing pole 30a is present in the gantry 11. The subject P stands upright with his or her back slightly leaning against the subject fixing pole 30a, so that a stable standing position posture can be maintained without fluctuations.

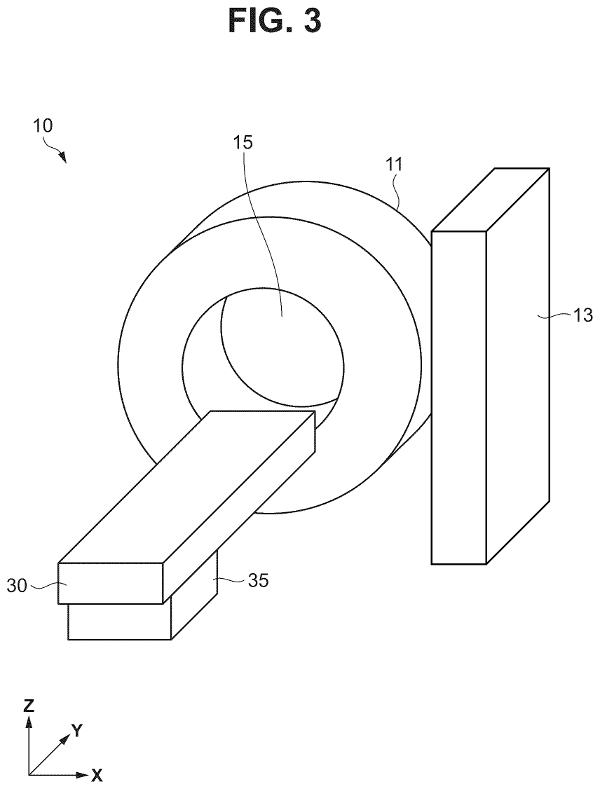

FIG. 3 is a perspective view illustrating an example of the state of the gantry apparatus 10 in the lying-position mode. As illustrated in FIG. 3, in the lying-position mode, the couchtop 30 is supported by a base 35 via the couchtop movement mechanisms 37 in a horizontal state. In this case, the couchtop 30 is freely movable along the major axis direction of the couchtop 30 under the control of the movement control circuitry 27.

The X-ray tube 17 is a vacuum tube that generates X-rays by radiating thermo-electrons from a cathode (filament) to an anode (target) by application of a high voltage from the high-voltage generator 31 and supply of a filament current. X-rays are generated due to collision of thermo-electrons on the target. The X-rays generated at the focal point of the tube in the X-ray tube 17 are formed in, for example, a cone beam shape via the collimator and are emitted to the subject P.

Examples of the X-ray tube 17 include a rotating anode X-ray tube that generates X-rays by emitting thermo-electrons onto a rotating anode. In the present embodiment, the X-ray tube 17 is also applicable to a single-tube X-ray CT apparatus and a so-called multi-tube X-ray CT apparatus having a configuration in which a plurality of pairs of X-ray tubes 17 and X-ray detectors 19 is mounted on the rotation frame 21.

The X-ray detector 19 detects an X-ray that is emitted from the X-ray tube 17 and has passed through the subject P, and outputs an electric signal corresponding to the amount of the X-ray to the DAS 33. The X-ray detector 19 includes, for example, a plurality of detection element arrays in which detection elements are arranged along a single arc about the focal point of the X-ray tube 17 in a channel direction. The X-ray detector 19 has a structure in which, for example, the plurality of detection element arrays is arranged in a slice direction (a column direction or a row direction).

Various types of the X-ray CT apparatus 1, including a rotate/rotate-type (third generation CT) having a configuration in which the X-ray tube 17 and the X-ray detector 19 rotate together around the subject P, and a stationary/rotate-type (fourth generation CT) having a configuration in which a large number of X-ray detection elements arrayed in a ring shape are fixed and the X-ray tube 17 alone rotates around the subject P, are applicable to the present embodiment. For the sake of specific explanation, a third generation CT will be described below as an example of the X-ray CT apparatus 1 according to the present embodiment.

The X-ray detector 19 is an indirect-conversion detector including a grid, a scintillator array, and an optical sensor array. The scintillator array includes a plurality of scintillators, and each scintillator includes a scintillator crystal that outputs light having a quantity of photon corresponding to an amount of incident X-rays. The grid is located on an X-ray incident side of the scintillator array and includes an X-ray shield plate having a function of absorbing scattered X-rays.

The grid may be referred to as a collimator (one-dimensional collimator or two-dimensional collimator). The optical sensor array has a function of converting an amount of light from the scintillators into a corresponding electric signal, and includes, for example, an optical sensor such as a photo multiplier (PMT).

The X-ray detector 19 may be a direct-conversion detector including a semiconductor element that converts an incident X-ray into an electric signal. Alternatively, the X-ray detector 19 may be a photon counting X-ray detector.

The rotation frame 21 includes the opening 15. The X-ray tube 17 that generates X-rays is attached to the rotation frame 21. Specifically, the rotation frame 21 is an annular frame that supports the X-ray tube 17 and the X-ray detector 19 in opposing positions to rotate the X-ray tube 17 and the X-ray detector 19 by the gantry control apparatus 25 to be described below.

The rotation frame 21 is supported rotatably about the main frame via a support bearing. The rotation frame 21 receives power from the rotation drive apparatus 23 under the control of the gantry control apparatus 25, and is rotated about the rotation axis A1 at a constant angular velocity about the rotation axis A1.

The rotation frame 21 includes not only the X-ray tube 17 and the X-ray detector 19, but also includes and supports the high-voltage generator 31 and the DAS 33. The rotation frame 21 having a configuration as described above is accommodated in a substantially cylindrical housing in which the opening 15 that forms the imaging space is formed. A central axis of the opening 15 coincides with the rotation axis A1 of the rotation frame 21.

Detected data generated by the DAS 33 is transmitted to, for example, a receiver that includes a photodiode and is provided on a non-rotational part (e.g., main frame) of the gantry apparatus 10 via optical communication from a transmitter including a light-emitting diode (LED), and is transferred to the console apparatus 100.

The method of transmitting detected data to the non-rotational part of the gantry apparatus 10 from the rotation frame 21 is not limited to the above-described optical communication, and any non-contact data transfer method may be employed.

The rotation drive apparatus 23 generates power for rotating the rotation frame 21 according to a control operation from the gantry control apparatus 25. The rotation drive apparatus 23 performs a driving operation at a rotational speed corresponding to a duty ratio or the like of a drive signal from the gantry control apparatus 25, thereby generating power. The rotation drive apparatus 23 is implemented by, for example, a motor such as a direct drive motor or a servo motor. The rotation drive apparatus 23 is accommodated in, for example, the gantry 11.

The gantry control apparatus 25 controls the high-voltage generator 31, the rotation drive apparatus 23, the movement control circuitry 27, and the DAS 33 according to a command from the console apparatus 100. The gantry control apparatus 25 has a function of receiving an input signal from an input interface attached to the console apparatus 100 or the gantry apparatus 10 and controlling the operation of the gantry apparatus 10.

For example, the gantry control apparatus 25 receives an input signal and performs a control operation for rotating the rotation frame 21, a control operation for tilting the gantry apparatus 10, and the like. The gantry control apparatus 25 may be provided on the post 13 in the gantry apparatus 10, or may be provided on the console apparatus 100. Each function to be implemented by the gantry control apparatus 25 may be implemented as a gantry control function in processing circuitry 107 in the console apparatus 100.

The gantry control apparatus 25 includes, as hardware resources, processing devices (processors), such as a central processing unit (CPU) and a micro processing unit (MPU), and storage devices (memories) such as a read only memory (ROM) and a random access memory (RAM).

The gantry control apparatus 25 may also be implemented by an application specific integrated circuit (ASIC), a field programmable gate array (FPGA), and any other complex programmable logic device (CPLD) or simple programmable logic device (SPLD).

The processing device reads out programs stored in the storage device and executes the programs, thereby implementing the functions described above. Instead of storing programs in the storage device, programs may be directly incorporated in the circuitry of the processing device. In this case, the processing device reads out programs incorporated in the circuitry and executes the programs, thereby implementing the functions described above.

The subject P can be placed on the couchtop 30 in the lying-position mode, and the couchtop 30 can be inserted into the opening 15. The couchtop 30 is supported on the base 35 via the couchtop movement mechanisms 37. Specifically, the couchtop 30 is held by the couchtop movement mechanisms 37 that are provided at the both ends in the Y-axis direction of the base 35.

The couchtop movement mechanism 37 enables the couchtop 30 to move along the direction in which the opening 15 penetrates. In other words, the couchtop 30 is fixed so as to be slidable and movable relative to the gantry 11 along the rotation axis A1 of the rotation frame 21 in the imaging system via the couchtop movement mechanism 37.

The couchtop movement mechanism 37 moves the couchtop 30 under the control of the movement control circuitry 27.

The couchtop movement mechanism 37 is composed of, for example, a roller guide or the like. The couchtop movement mechanism 37 can be implemented by frictional driving or a configuration such as a belt mechanism. The couchtop movement mechanism 37 is not limited to a roller guide, frictional driving, a belt mechanism, or the like, but instead may be implemented by an existing mechanism as appropriate.

The couchtop movement mechanism 37 may be mounted on an up-and-down motion mechanism. The up-and-down motion mechanism is mounted on, for example, the couchtop movement mechanism 37 and is provided on the base 35. The up-and-down motion mechanism can move the couchtop 30 in a direction perpendicular to a surface on which the subject P is placed on the couchtop 30.

For example, the up-and-down motion mechanism can be implemented by an actuator (e.g., piston type) configured to move (push up) the rotation shaft of the roller guide along the Y-axis direction. The up-and-down motion mechanism is not limited to an actuator, but instead may be implemented any other mechanism.

A left-to-right motion mechanism may be provided between the couchtop movement mechanism 37 and the couchtop 30. For example, couchtop support members that cover the lower surface and the side surface of the couchtop 30 are provided on the lower surface of the couchtop 30 and the side surface of the couchtop 30. The left-to-right motion mechanism includes a block, a ball screw, a motor, and a belt. The ball screw stretches along the minor axis direction of the couchtop 30. The block is attached to the ball screw.

The block is connected to couchtop support members. A torque from the motor is transmitted to the ball spring via the belt. When the motor is rotated under the control of the movement control circuitry 27, the torque from the motor is transmitted to the ball screw. This allows the ball screw to rotate. Along with the rotation of the ball screw, the block moves along the minor axis direction of the couchtop 30.

The movement control circuitry 27 controls the movement of each of the gantry 11 and the couchtop 30.

For example, if an instruction to capture an image of the subject P in the standing-position mode is issued from a user, the movement control circuitry 27 controls the gantry movement mechanism 131 to rotate the gantry 11 so that the opening 15 is oriented in the vertical direction.

For example, if an instruction to capture an image of the subject P in the standing-position mode is issued from the user, the movement control circuitry 27 controls the couchtop movement mechanism 37 to evacuate the couchtop 30 to a position where the couchtop 30 does not interfere with the gantry 11 even when the gantry 11 is moved along the vertical direction.

The base 35 may be provided with a movement mechanism such as a caster. In this case, the base 35 may be manually moved by the user to evacuate the couchtop 30, or the base 35 may be automatically moved by a drive source such as a motor to evacuate the couchtop 30.

The movement control circuitry 27 can be implemented by a processor or the like as described above.

While FIG. 1 illustrates an example where the movement control circuitry 27 is mounted on the post 13, the movement control circuitry 27 may be mounted on the gantry 11 or may be mounted on the console apparatus 100. Each function to be implemented by the movement control circuitry 27 may be mounted on the processing circuitry 107 or may be mounted on the gantry control apparatus 25 as a movement control function.

An operation panel 29 is implemented by a switch button, a touch pad for performing an input operation by touching on an operation surface, a touch panel display having a configuration in which a display screen and a touch pad are integrated together, or the like. The operation panel 29 converts an input operation received from the user into an electric signal, and outputs the electric signal to the gantry control apparatus 25.

The operation panel 29 receives a selection operation of selecting an imaging protocol including, for example, a standing-position mode related to imaging of the subject P in the standing position posture, a seated-position mode related to imaging of the subject P in the seated position posture, or a lying-position mode related to imaging of the subject P in the lying position posture. The operation panel 29 is provided on, for example, the post 13.

The high-voltage generator 31 includes electric circuitry such as a transformer and a rectifier, and generates a high voltage to be applied to the X-ray tube 17 and a filament current to be supplied to the X-ray tube 17. The high-voltage generator 31 controls an output voltage based on X-rays emitted from the X-ray tube 17. The high-voltage generator 31 may be a transformer type generator or an inverter type generator.

The high-voltage generator 31 may be provided on the rotation frame 21, or may be provided on the main frame of the gantry 11.

The wedge (not illustrated) is a filter for adjusting the amount of X-rays emitted from the X-ray tube 17. Specifically, the wedge is a filter that allows the X-rays emitted from the X-ray tube 17 to transmit therethrough for attenuation, so that the subject P is irradiated with the X-rays from the X-ray tube 17 in a predetermined distribution.

The wedge is, for example, a wedge filter or a bow-tie filter, and is formed by processing aluminum with a predetermined target angle and a predetermined thickness.

The collimator (not illustrated) includes a combination of a plurality of lead plates, which form a slit, to converge the X-rays having passed through the wedge in an irradiation range.

The DAS 33 includes an amplifier for performing amplification processing on an electric signal output from each X-ray detection element of the X-ray detector 19, and an analog-to-digital (A/D) converter for converting an electric signal into a digital signal, and generates detected data. The detected data generated by the DAS 33 is transferred to the console apparatus 100.

The console apparatus 100 includes a memory 101, a display 103, an input interface 105, and the processing circuitry 107. Data communication between the memory 101, the display 103, the input interface 105, and the processing circuitry 107 is performed via, for example, a bus.

The memory 101 is a storage device for storing various information, such as a hard disk drive (HDD), a solid state drive (SSD), or an integrated circuit storage device. For example, the memory 101 stores projection data and reconstruction image data.

For example, the memory 101 stores parameter information 1011. The parameter information 1011 is stored by associating a dose parameter with information indicating which one of the lying-position mode and the standing-position mode is set as an imaging mode. The parameter information 1011 is used for the processing circuitry 107 to be described below to perform dose parameter determination processing and output dose calculation processing using the dose parameter.

The memory 101 is not limited to an HDD, an SSD, or the like, but instead may be a drive apparatus configured to read and write various information from and to portable storage media such as a compact disc (CD), a digital versatile disc (DVD), and a flash memory, semiconductor memory elements such as a RAM, and the like.

A storage region of the memory 101 may be located within the console apparatus 100, or may be located within an external storage device connected via a network. The memory 101 stores control programs according to the present embodiment. The memory 101 stores volume data and the like generated by pre-scan or main-scan processing.

The display 103 displays various types of information. For example, the display 103 outputs medical images (CT images) generated by the processing circuitry 107, a graphical user interface (GUI) for receiving various operations from the user, and the like.

As the display 103, for example, a liquid crystal display (LCD), a cathode ray tube (CRT) display, an organic electro luminescence display (OELD), a plasma display, or any other display can be used as appropriate.

The display 103 may be provided on the gantry apparatus 10. The display 103 may be a desktop display, or may be composed of a tablet terminal or the like configured to establish wireless communication with the main body of the console apparatus 100. The display 103 corresponds to a display unit.

The input interface 105 receives various input operations from the user, converts the received input operations into electric signals, and outputs the electric signals to the processing circuitry 107. For example, the input interface 105 receives acquisition conditions for acquiring projection data, reconstruction conditions for reconstructing a CT image, image processing conditions for generating a post-processing image based on the CT image, and the like from the user.

As the input interface 105, for example, a mouse, a keyboard, a trackball, a switch, a button, a joystick, a touch pad, or a touch panel display can be used.

In the present embodiment, the input interface 105 is not limited to input interfaces including physical operation members such as a mouse, a keyboard, a trackball, a switch, a button, a joystick, a touch pad, and a touch panel display.

Examples of the input interface 105 also include processing circuitry that receives an electric signal corresponding to an input operation from an external input device provided separately from the apparatus and outputs the electric signal to processing circuitry 107.

The input interface 105 may be provided on the gantry apparatus 10. The input interface 105 may be composed of a tablet terminal or the like configured to establish wireless communication with the main body of the console apparatus 100. The input interface 105 corresponds to an input unit.

The processing circuitry 107 controls an overall operation of the X-ray CT apparatus 1 according to an electric signal corresponding to an input operation output from the input interface 105. For example, the processing circuitry 107 includes, as hardware resources, a processor such as a CPU, an MPU, or a graphics processing unit (GPU), and a memory such as a ROM or a RAM.

The processing circuitry 107 causes the processor configured to execute programs loaded into the memory to execute a system control function 111, a determination function 113, a calculation function 115, a pre-processing function 117, a reconstruction function 119, and an image processing function 121.

In this case, the system control function 111 is an example of a reception unit. The determination function 113 is an example of each of an identification unit and a determination unit. The calculation function 115 is an example of a calculation unit.

Each of the system control function 111, the determination function 113, the calculation function 115, the pre-processing function 117, the reconstruction function 119, and the image processing function 121 need not necessarily be implemented by a single processing circuitry. Processing circuitry may be formed by combining a plurality of independent processors and each processor may execute programs to thereby implement the system control function 111, the determination function 113, the calculation function 115, the pre-processing function 117, the reconstruction function 119, and the image processing function 121.

The system control function 111 controls each function of the processing circuitry 107 based on an input operation received from the user via the input interface 105.

Specifically, the system control function 111 reads out control programs stored in the memory 101 and loads the control programs into the memory in the processing circuitry 107, thereby controlling each unit of the X-ray CT apparatus 1 according to the loaded control programs. For example, the system control function 111 controls each function of the processing circuitry 107 based on an input operation received from the user via the input interface 105.

In the present embodiment, the input operation includes a selection input of an imaging protocol. The imaging protocol includes information indicating which one of the standing-position mode and the lying-position mode is set as the imaging mode.

The determination function 113 determines a dose parameter used to calculate an output dose based on the type of an object present on an X-ray tube path (between the X-ray tube 17 and the X-ray detector 19).

In this case, an object present on the X-ray tube path during imaging of the subject P in the lying-position mode is different from an object present on the X-ray tube path during imaging of the subject P in the standing-position mode, to be more specific, the couchtop 30 is present on the X-ray tube path during imaging of the subject P in the lying-position mode, while the subject fixing pole 30a is present on the X-ray tube path during imaging of the subject P in the standing-position mode.

For this reason, even in a case where the output dose is calculated using the same dose parameter when an image of the subject P is captured under the same imaging conditions except for the imaging mode in the same X-ray CT apparatus 1, an exposure dose of the subject P when the standing-position mode is set as the imaging mode may be different from that when the lying-position mode is set as the imaging mode. In other words, there is a possibility that the output dose to be calculated cannot be adjusted to an optimum value depending on the imaging mode.

Accordingly, the X-ray CT apparatus 1 according to the present embodiment stores an optimum dose parameter by taking into consideration an object present on the X-ray tube path for each imaging mode and uses the dose parameter suitable for the imaging mode, thereby adjusting the output dose to be calculated to an optimum value.

For example, the determination function 113 identifies which one of the standing-position mode and the lying-position mode is set as the imaging mode based on information included in the selected and input imaging protocol received by the system control function 111. The determination function 113 refers to the parameter information 1011 in the memory 101, and determines the dose parameter associated with the identified imaging mode to be the dose parameter used to calculate the output dose.

The calculation function 115 calculates the output dose using the dose parameter determined by the determination function 113.

For example, in a state where no X-rays are emitted from the X-ray tube 17, the calculation function 115 uses the dose parameter determined by the determination function 113, and calculates a predicted value of the output dose in a case where an image of the subject P is captured under imaging conditions including a tube voltage and a tube current corresponding to the imaging protocol received by the system control function 111.

For example, in a state where X-rays are emitted from the X-ray tube 17, the calculation function 115 calculates the output dose from the start of X-ray irradiation to the present time by using the does parameter determined by the determination function 113. The calculation result is output in such a manner that, for example, the calculation result is displayed on the GUI by the system control function 111.

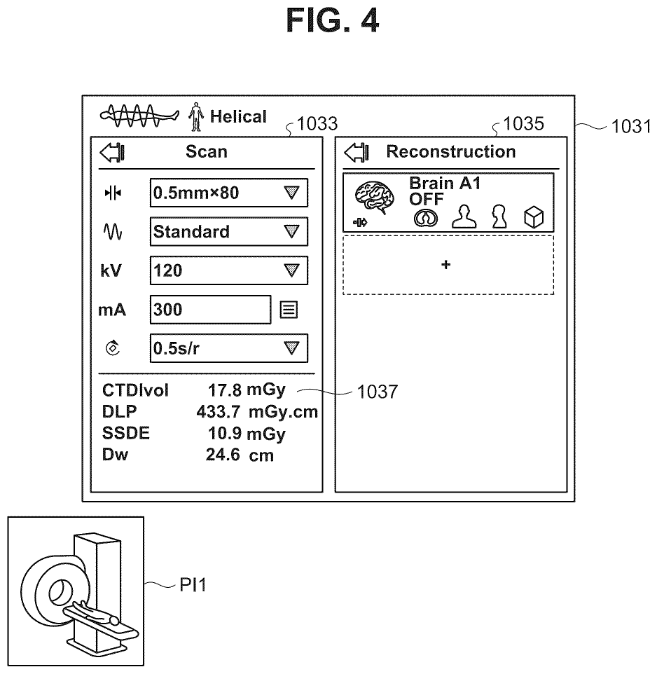

FIG. 4 illustrates an example of an output dose display screen 1031 when an image of the subject P is captured in the lying-position mode.

The display screen 1031 illustrated in FIG. 4 is a screen example where the output dose in a case where an image of the subject P is captured in the lying-position mode is calculated and displayed on the GUI for receiving various operations from the user. For convenience of explanation, a captured image PI1 is illustrated in FIG. 4 to indicate that the lying-position mode is set as the imaging mode for the subject P. The captured image PI1 may be displayed together with the display screen 1031.

The display screen 1031 includes, as screen components, an imaging conditions setting field 1033, a reconstruction conditions setting field 1035, and an output dose display field 1037.

The imaging conditions setting field 1033 is a display field for operation elements to receive an input operation for inputting various imaging conditions (e.g., an imaging range, a tube voltage, a tube current, and an X-ray tube rotational speed). In the present embodiment, when the system control function 111 receives a selection input of an imaging protocol from the user, various imaging conditions corresponding to the imaging protocol are automatically displayed. The user can change and adjust various imaging conditions based on various imaging conditions automatically displayed.

The reconstruction conditions setting field 1035 is a display field for operation elements to receive an input operation for inputting reconstruction conditions (e.g., designation of an organ to be imaged) used for reconstruction processing. In the present embodiment, when the system control function 111 receives a selection input of an imaging protocol from the user, reconstruction conditions corresponding to the imaging protocol are automatically displayed. The user can change and adjust reconstruction conditions based on the reconstruction conditions automatically displayed.

The output dose display field 1037 is a display field for displaying the output dose calculated by the calculation function 115 using the dose parameter determined by the determination function 113. In the example illustrated in FIG. 4, the output dose calculated using the dose parameter associated with information indicating the lying-position mode in the parameter information 1011 is displayed on the output dose display field 1037.

In the example illustrated in FIG. 4, CTDIvol, DLP, and Size-Specific Dose Estimates (SSDE) are displayed as examples of the output dose.

CTDIvol represents an absorbed dose of radiation per 1 cm on the subject P. DLP represents an absorbed dose of radiation corresponding to an imaging range. DLP is calculated by an expression “CTDIvol×imaging range (cm)”. SSDE represents a dose index obtained by taking into consideration a difference in the body type of each subject P. SSDE is used to evaluate an approximate absorbed dose on an arbitrary cross-section.

On the output dose display field 1037, information indicating a water-equivalent diameter Dw used to calculate SSDE is also displayed as information related to the output dose.

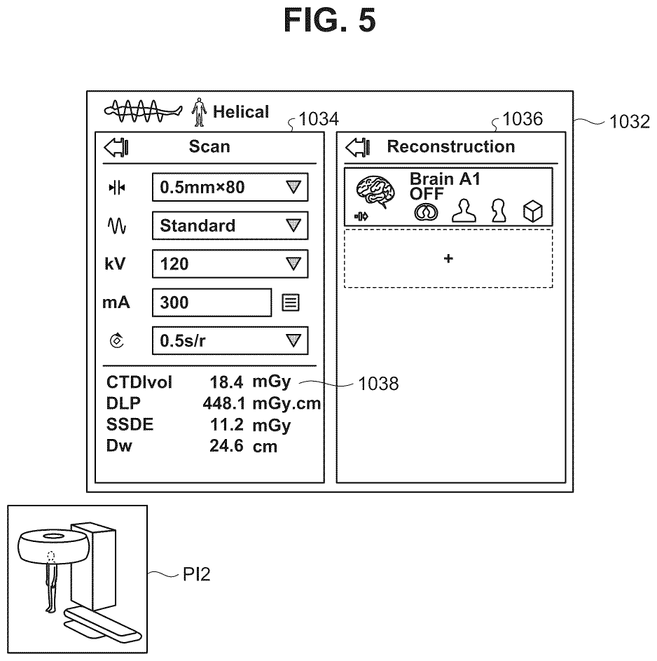

FIG. 5 illustrates an example of an output dose display screen 1032 when an image of the subject P is captured in the standing-position mode.

The display screen 1032 illustrated in FIG. 5 is a screen example where the output dose in a case where an image of the subject P is captured in the standing-position mode is calculated and displayed on the GUI for receiving various operations from the user. For convenience of explanation, a captured image PI2 is illustrated in FIG. 5 to indicate that the standing-position mode is set as the imaging mode for the subject P. The captured image PI2 may be displayed together with the display screen 1032.

The display screen 1032 includes, as screen components, an imaging conditions setting field 1034, a reconstruction conditions setting field 1036, and an output dose display field 1038. The imaging conditions setting field 1034 and the reconstruction conditions setting field 1036 are similar to the imaging conditions setting field 1033 and the reconstruction conditions setting field 1035, respectively, and thus descriptions thereof are omitted.

In the example illustrated in FIG. 5, the output dose calculated using the dose parameter associated with information indicating the standing-position mode in the parameter information 1011 is displayed on the output dose display field 1038.

As illustrated in FIGS. 4 and 5, according to the present embodiment, in a case where different imaging modes are set, different values are displayed as the output dose on the GUI even when an image of the subject P is captured under the same imaging conditions. Accordingly, the output dose to be calculated for each imaging mode is adjusted to an optimum value, which enables the user to manage the radiation dose with higher accuracy.

While the present embodiment described above illustrates a configuration in which the system control function 111 controls display of the calculated output dose on the GUI, the method of outputting the output dose is not limited to the above-described method. For example, the system control function 111 may output the calculated output dose to an examination summary, DoseSR, or the like.

Referring again to FIG. 1, the description of the configuration of the X-ray CT apparatus 1 will be continued. The pre-processing function 117 generates data by performing pre-processing, such as logarithmic transformation processing, offset correction processing, inter-channel sensitivity correction processing, or beam hardening correction, on raw data output from the DAS 33. Data obtained before pre-processing is also referred to as raw data, and data obtained after pre-processing is also referred to as projection data.

The reconstruction function 119 executes reconstruction processing on projection data, thereby generating a reconstruction image.

For example, the reconstruction function 119 performs reconstruction processing using a filtered back projection (FBP) method, a successive approximation reconstruction method, or the like on the projection data generated by the pre-processing function 117, thereby generating CT image data. In other words, the reconstruction function 119 generates a reconstruction image based on an output from the imaging system. The reconstruction function 119 stores the generated reconstruction image data in the memory 101.

The image processing function 121 performs various types of image processing on the reconstruction image generated by the reconstruction function 119. For example, the image processing function 121 generates a display image by performing three-dimensional image processing, such as volume rendering, surface volume rendering, image value projection processing, multi-planer reconstruction (MPR) processing, or curved MPR (CPR) processing, on the CT image.

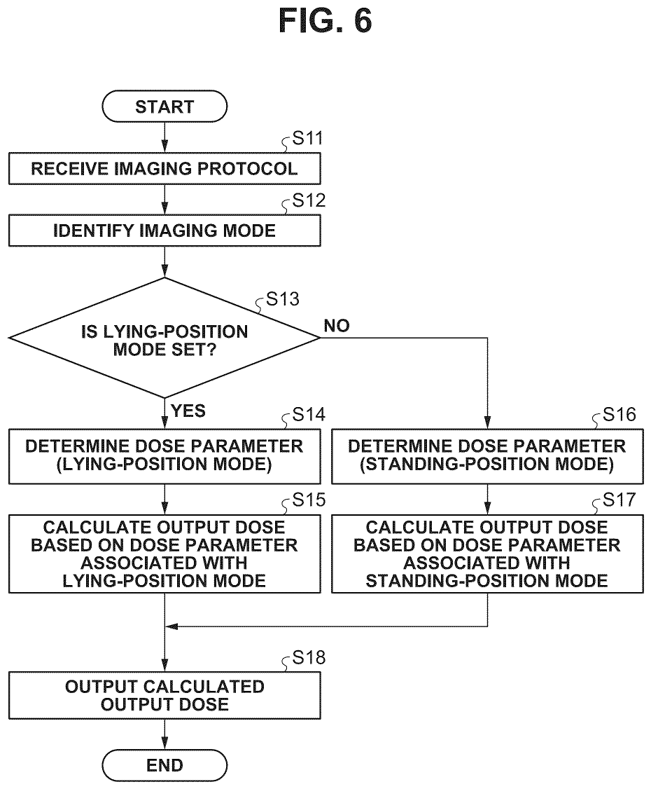

Processing to be executed by the X-ray CT apparatus 1 according to the present embodiment will be described below. FIG. 6 is a flowchart illustrating an example of processing to be executed by the X-ray CT apparatus 1 according to the embodiment.

FIG. 6 illustrates an example where a predicted value of the output dose is calculated and output in a case where an image of the subject P is captured under imaging conditions corresponding to the imaging protocol received by the system control function 111 before imaging of the subject P. However, the processing of calculating and outputting the output dose may be executed during imaging of the subject P or after imaging of the subject P.

First, in step S11, the system control function 111 receives a selection input of an imaging protocol from the user. The imaging protocol includes information indicating which one of the lying-position mode and the standing-position mode is set as the imaging mode.

Next, in step S12, the determination function 113 identifies the imaging mode for the subject P. For example, the determination function 113 identifies the imaging mode for the subject P based on the information indicating which one of the lying-position mode and the standing-position mode is set as the imaging mode included in the imaging protocol received in step S11. Next, in step S13, the determination function 113 determines whether the identified imaging mode is the lying-position mode (standing-position mode or seated-position mode).

If the lying-position mode is set as the imaging mode (YES in step S13), the processing proceeds to step S14. In step S14, the determination function 113 refers to the parameter information 1011 in the memory 101 and determines the dose parameter associated with the lying-position mode to be the dose parameter used to calculate the output dose.

Next, in step S15, the calculation function 115 calculates the output dose based on the dose parameter associated with the lying-position mode. For example, the calculation function 115 calculates the output dose using the imaging conditions corresponding to the imaging protocol received in step S11 and the dose parameter associated with the lying-position mode determined in step S14. After step S15, the processing proceeds to step S18 to be described below.

On the other hand, if the standing-position mode or the seated-position mode is set as the imaging mode (NO in step S13), the processing proceeds to step S16. In step S16, the determination function 113 refers to the parameter information 1011 in the memory 101 and determines the dose parameter associated with the standing-position mode to be the dose parameter used to calculate the output dose.

Next, in step S17, the calculation function 115 calculates the output dose based on the dose parameter associated with the standing-position mode. For example, the calculation function 115 calculates the output dose using the imaging conditions corresponding to the imaging protocol received in step S11 and the dose parameter associated with the standing-position mode determined in step S16.

Next, in step S18, the system control function 111 outputs the output dose, and then the processing ends. For example, the system control function 111 controls display of the output dose calculated in step S15 or the output dose calculated in step S17 on the GUI.

As described above, the X-ray CT apparatus 1 according to the present embodiment identifies the posture of the gantry 11 and the object present on the X-ray path, determines the dose parameter used to calculate the output dose based on the identified posture of the gantry 11, the object present on the X-ray path, and the parameter information 1011 to be stored by associating the posture of the gantry 11 and the object present on the X-ray path with the dose parameter used to calculate the output dose to be output from the X-ray CT apparatus 1, and calculates the output dose using the determined dose parameter.

For example, if the output dose is calculated using the same dose parameter regardless of the state of the gantry 11, there may be a difference between the output dose to be calculated and the actual output dose depending on the state of the gantry 11. This is because a different object (couchtop 30 or subject fixing pole 30a) is present on the X-ray path depending on the state of the gantry 11 and the amount of absorption of X-rays varies depending on the object. On the other hand, the X-ray CT apparatus 1 according to the present embodiment can calculate the output dose using the dose parameter suitable for each type of objects present on the X-ray path. With this configuration, the X-ray CT apparatus 1 according to the present embodiment can adjust the output dose to be calculated to an optimum value for the state of the gantry 11 (the type of the object present on the X-ray path).

The above-described embodiment can be modified as appropriate by changing a part of the configuration of the X-ray CT apparatus 1 or some of the functions of the X-ray CT apparatus 1. Accordingly, modified examples of the above-described embodiment will be described below as other embodiments. In the following description, differences from the above-described embodiment will be mainly described, and detailed descriptions of the same contents as those described above will be omitted. The following modified examples may be individually carried out, or may be carried out in combination as appropriate.

### Modified Example 1

The embodiment described above illustrates a configuration in which processing of determining a dose parameter and processing of calculating an output dose are executed assuming that the standing-position mode is set as the imaging mode if the seated position is set as the imaging posture of the subject P, like in the case where the standing position is set as the imaging posture of the subject P. In Modified Example 1, a configuration in which the dose parameter in the seated-position imaging state (also referred to as the seated-position mode) is stored and processing of calculating the output dose based on the dose parameter corresponding to the seated-position mode is executed if the seated position is set as the imaging posture of the subject P will be described.

In Modified Example 1, the parameter information 1011 in the memory 101 is stored by associating information indicating that the seated-position mode is set as the imaging mode with an optimum dose parameter for the seated-position mode.

In Modified Example 1, the imaging protocol includes information indicating which one of the lying-position mode, the standing-position mode, and the seated-position mode is set as the imaging mode.

If the seated-position mode is set as the imaging mode included in the imaging protocol received by the system control function 111, the determination function 113 refers to the parameter information 1011 in the memory 101 and determines the dose parameter associated with the seated-position mode to be the dose parameter used to calculate the dose amount.

In Modified Example 1, if the seated position is set as the imaging posture of the subject P, the dose parameter that is suitable for the seated-position mode and is different from the dose parameter corresponding to the standing-position mode can be used to calculate the output dose. In other words, according to Modified Example 1, the output dose to be calculated can be adjusted to an optimum value.

### Modified Example 2

The embodiment described above illustrates a configuration in which the imaging posture is one of the lying position (an installation surface of the post 13 is parallel to the axis of the body of the subject P), the standing position, and the seated position (the installation surface of the post 13 is vertical to the axis of the body of the subject P). In Modified Example 2, a configuration in which an image of the subject P can be captured by setting an angle formed between the installation surface of the post 13 and the axis of the body of the subject P to a desired angle within a range from 90° (the installation surface of the post 13 is vertical to the axis of the body of the subject P) to 180° (the installation surface of the post 13 is parallel to the axis of the body of the subject P) will be described.

In Modified Example 2, the couchtop movement mechanism 37 includes a mechanism configured to change the angle formed between the surface on which the subject P is placed on the couchtop 30 and the installation surface of the post 13 to a desired angle within the range from 90° to 180°. The system control function 111 according to Modified Example 2 receives an input operation for inputting a desired angle of the couchtop 30 (also referred to as a couchtop angle) with respect to the installation surface of the post 13 from the user. For example, the system control function 111 receives an input operation for inputting a desired angle within the range from 90° to 180° in units of 5°.

The movement control circuitry 27 according to Modified Example 2 performs a control operation for the user to change the angle of the couchtop 30 (hereinafter also referred to as the couchtop angle) with respect to the installation surface of the post 13 to a desired angle within the range from 90° to 180°.

When the user changes the angle of the couchtop 30 to a desired angle within the range from 90° to 180°, the movement control circuitry 27 causes the gantry 11 to rotate so that the opening 15 can be vertical to the surface on which the subject P is placed on the couchtop 30.

In Modified Example 2, the parameter information 1011 in the memory 101 is stored by associating the optimum dose parameter with the angle of the couchtop 30 for each angle of the couchtop 30 (every 5° within the range from 90° to 180° in Modified Example 2).

In Modified Example 2, the imaging protocol includes information indicating the angle of the couchtop 30 at which an image of the subject P is to be captured.

The determination function 113 refers to the parameter information 1011 in the memory 101 and determines the dose parameter associated with information indicating the angle of the couchtop 30 included in the imaging protocol received by the system control function 111 to be the dose parameter used to calculate the output dose.

According to Modified Example 2, an optimum dose parameter for the angle of the couchtop 30 can be used to calculate the output dose. In other words, according to Modified Example 2, the output dose to be calculated can be adjusted to an optimum value.

### Modified Example 3

The embodiment described above illustrates a configuration in which an optimum dose parameter for each imaging mode is preliminarily determined for each imaging mode. In Modified Example 3, a configuration in which an optimum calculation method for each imaging mode (for example, multiplying a correction coefficient prepared for each imaging mode after calculation of an output dose) is determined for each imaging mode using the same dose parameter regardless of the imaging mode will be described.

In Modified Example 3, one dose parameter is stored as the parameter information 1011 in the memory 101. Further, the memory 101 stores calculation information. The calculation information is stored by associating information indicating the imaging mode with the correction coefficient. In this case, the correction coefficient is determined by taking into consideration the type of the object (couchtop 30 or subject fixing pole 30a) present on the X-ray tube path.

In Modified Example 3, assume that the system control function 111 executes storage processing by associating information indicating the imaging mode with the correction coefficient. In this case, the system control function 111 is an example of a storage control unit.

In Modified Example 3, the calculation function 115 identifies the imaging mode for capturing an image of the subject P based on information included in the imaging protocol received by the system control function 111. The calculation function 115 refers to the calculation information in the memory 101 and identifies the correction coefficient associated with the identified imaging mode.

The calculation function 115 calculates the output dose based on the dose parameter stored as the parameter information 1011 in the memory 101, and uses the value obtained by multiplying the calculation result by the identified correction coefficient as the output dose.

According to Modified Example 3, there is no need to prepare a dose parameter for each imaging mode, which leads to a reduction in the burden of work for preparing the dose parameter.

### Modified Example 4

The embodiment described above illustrates a configuration in which the type of the object present on the X-ray tube path is the couchtop 30 or the subject fixing pole 30a. In Modified Example 4, a configuration in which an image of the subject P is captured in a state where an object other than the couchtop 30 or the subject fixing pole 30a is inserted into the opening 15 of the gantry 11 will be described.

In Modified Example 4, during imaging of the subject P in the lying-position mode, not only the couchtop 30 (CT couch), but also an angio couch, a flat couchtop, and an operating table are used. These are examples of the type of the couch used for imaging of the subject P in the lying-position mode. Any other type of couch (couchtop) may also be used for imaging of the subject P.

In Modified Example 4, the parameter information 1011 in the memory 101 is stored by associating imaging conditions, information indicating an object present on the X-ray tube path, and a dose parameter.

In Modified Example 4, the imaging protocol includes information indicating the object present on the X-ray tube path. The information indicating the type of the object present on the X-ray tube path is an example of each of object information and gantry information.

The determination function 113 identifies information indicating the object present on the X-ray tube path based on the information included in the imaging protocol received by the system control function 111.

The determination function 113 refers to the parameter information 1011 in the memory 101 and determines the dose parameter associated with the identified information indicating the object present on the X-ray tube path to be the dose parameter used to calculate the output dose.

A plurality of objects may be present on the X-ray tube path. Examples of the objects present on the X-ray tube path may also include clothes worn by the subject P and drip tubing.

In Modified Example 4, even in a case where an image of the subject P is captured by inserting an object other than the couchtop 30 or the subject fixing pole 30a into the opening 15 of the gantry 11, an optimum dose parameter for the imaging mode can be used to calculate the output dose. In other words, according to Modified Example 4, the output dose to be calculated can be adjusted to an optimum value.

According to at least one of the above-described embodiments, modified examples, and the like, an output dose to be calculated can be adjusted to an optimum value in an X-ray CT apparatus configured to capture an image of a subject in a lying position state or a standing position state.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography (CT) apparatus comprising processing circuitry configured to:
identify gantry state information indicating a state of a gantry including an opening, the gantry state information including at least one of posture information indicating a posture of the gantry and object information indicating an object other than a subject present in the opening;
determine a dose parameter used to calculate an output dose based on the identified gantry state information and parameter information to be stored by associating the gantry state information with the dose parameter used to calculate the output dose to be output from the X-ray CT apparatus; and
calculate the output dose using the determined dose parameter.

2. The X-ray CT apparatus according to claim 1,
wherein the gantry state information includes the object information, and
wherein the processing circuitry is further configured to store the parameter information by associating the object information indicating a type of the object with the dose parameter for each type of the object, at least a part of the dose parameter being different in each type of the object.

3. The X-ray CT apparatus according to claim 2, wherein the object includes a couchtop on which the subject in a lying position state is placed during imaging, and a columnar subject holder configured to support the subject in a standing position state during imaging.

4. The X-ray CT apparatus according to claim 3, wherein the processing circuitry performs one of processing for determining a calculation method using the dose parameter associated with another object including the subject holder based on the dose parameter associated with the couchtop and storing the parameter information by associating the calculation method with information indicating a type of the object, and processing for determining a calculation method using the dose parameter associated with another object including the couchtop based on the dose parameter associated with the subject holder and storing the parameter information by associating the calculation method with information indicating a type of the object.

5. The X-ray CT apparatus according to claim 3,
wherein the X-ray CT apparatus is configured to perform Angio-CT imaging on the subject, and
wherein the object further includes an angio couch on which the subject is placed during Angio-CT imaging of the subject.

6. The X-ray CT apparatus according to claim 1, wherein the processing circuitry is further configured to:
receive an input of at least imaging conditions for imaging of the subject and an imaging protocol including the gantry state information, and
identify the gantry state information based on the received imaging protocol.

7. An information processing method for calculating an output dose to be output from an X-ray CT apparatus, the information processing method comprising:
identifying gantry state information indicating a state of a gantry including an opening into which a subject is inserted, the gantry state information including at least one of posture information indicating a posture of the gantry and object information indicating an object other than the subject present in the opening;
determining a dose parameter used to calculate the output dose based on the identified gantry state information and parameter information to be stored by associating the gantry state information with the dose parameter used to calculate the output dose to be output from the X-ray CT apparatus; and
calculating the output dose using the determined dose parameter.
